(19) United States
(12) Patent Application Publication
(10) Pub. No.: US 2024/0000855 A1
(43) Pub. Date: Jan. 4, 2024

(54) SPECIFIC PATHOGEN FREE FEED COMPOSITION

(71) Applicant: BASF SE, Ludwigshafen am Rhein (DE)

(72) Inventors: Arnulf TROESCHER, Ludwigshafen (DE); Simon CHAMP, Ludwigshafen (DE); Jörg RIESMEIER, Düsseldorf (DE)

(21) Appl. No.: 18/039,690

(22) PCT Filed: Dec. 2, 2021
(86) PCT No.: PCT/EP2021/083859

§ 371 Date: May 31, 2023

(30) Foreign Application Priority Data
Dec. 2, 2020 (EP) 20211217.3
Feb. 23, 2021 (EP) 21158669.8

(51) Int. Cl. A61K 35/62; A23K 10/20; A23K 50/75; A23K 50/60; A61P 17/02

(52) U.S. Cl. CPC A61K 35/62; A23K 10/20; A23K 50/75; A23K 50/60; A61P 17/02; A01K 67/0332

(57) ABSTRACT

The presently claimed invention is directed to a feed composition comprising at least one living worm, wherein the worm is free of pathogens selected from the group consisting of species of Salmonella, species of Campylobacter, Histomonas meleagridis and any combination of two or more of the aforementioned.

### FIELD OF INVENTION

The presently claimed invention is directed to a feed composition comprising at least one living worm, wherein the worm is free of pathogens selected from the group consisting of species of Salmonella, species of Campylobacter, Histomonas meleagridis and any combination of two or more of the aforementioned.

### BACKGROUND OF INVENTION

Complete protein is a food source of protein which contains all of the essential amino acids in adequate proportion for the human diet. Among the complete proteins, meat, eggs and milk are considered to be among the highest-quality protein sources. Among these, meat, especially from the cattle and poultry industry, is considered as one of the most important sources of complete protein. In the poultry industry, broiler chickens are the animals most relied on as source of meat and therefore as complete proteins.

In modern broiler production, chickens hatch in artificial incubators and are reared under environmental conditions with high hygienic standards. Due to these high hygienic standards a proper exposure to microorganism, particularly to those with beneficial effects is lacking. Additionally, the lack of mother-offspring interactions impairs the acquisition of initial gut flora from or through the mother.

Earthworms belong to natural feed sources for avian species, including chickens. Since earthworms are dependent on microorganisms for the digestion of cell wall constituents of plant materials and have a relatively high microbial activity in their gut, they may be considered as ‘natural’ microbiota-inoculates for chicken when fed alive. However, this may lead to inoculation of harmful microbiota too.

One problem faced in the poultry production is the high rate of microbial infection leading to high rates of death especially among young animals. Thus, the industry is forced to provide antibiotics to the young animals which in turn indirectly leads to an increased antibiotic resistance in humans. Thus, it is desirable to avoid the feeding of antibiotics to young animals yet protect them from the microbial infections.

Accordingly, the main object of the presently claimed invention is to provide a feed composition that can provide the positive microbiota to the animal and additionally to provide improved protection from the microbial infections.

Another object of the presently claimed invention is to provide a feed composition which is free of pathogens selected from the group consisting of species of Salmonella, species of Campylobacter, Histomonas meleagridis and any combination of two or more of the aforementioned.

Yet another object of the presently claimed invention is to provide a feed composition that is able to maintain animal free of specific pathogen (SPF), especially chicken according to Ph. Eur. 9, Ausgabe, Grundwerk 2017, 9.0/5020200.

Another object of the presently claimed invention is to provide a method for producing vermicompost which is free of pathogens selected from the group consisting of species of Salmonella, species of Campylobacter, Histomonas meleagridis and any combination of two or more of the aforementioned and/or SPF according to Ph. Eur. 9, Ausgabe, Grundwerk 2017, 9.0/5020200.

Another object of the presently claimed invention is to provide a method to transfer positive microbiota to animals, which leads to increased counts of positive microbiota in the feces.

### SUMMARY OF THE INVENTION

Surprisingly, it was found that feeding young animals with feed composition comprising at least one living worm,
wherein the worm is free of pathogens selected from the group consisting of species of Salmonella, species of Campylobacter, Histomonas meleagridis and any combination of two or more of the aforementioned, provides/increases the positive microbiota in the animal and additionally provides improved protection from microbial infections.

Accordingly, the first aspects of the presently claimed invention is directed to a feed composition comprising
at least one living worm,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 90/5020200.

Second aspects of the presently claimed invention is directed to a feed composition comprising:
a. vermicompost in an amount in the range of 10 wt. % to 90 wt. % based on the total wet weight of the composition; and
b. worms in an amount in the range of 10 wt. % to 90 wt. % based on the total wet weight of the composition.
   wherein the vermicompost is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 90/502.0200.

The third aspect of the presently claimed invention is directed to a process for feeding animals for non-therapeutic use comprising
feeding the animal with a composition in an amount in the range of 2 mg to 50 mg living worm/g body weight of the animal per day, wherein the composition comprises at least one living worm and wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

The fourth aspect of the presently claimed invention is directed to a method of producing vermicompost comprising the steps of:
a. raising worms from pathogen free breeding stock /parents;
b. feeding the worms with pathogen free feed;
c. keeping/raising the worms in pathogen free surrounding; and
d. collecting the vermicompost,
   wherein “pathogen free” is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5020200.

The fifth aspect of the presently claimed invention is directed to a pathogen free vermicompost, wherein “pathogen free” is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

The sixth aspect of the presently claimed invention is directed to a process for preparing a liquid vermicompost extract comprising the steps of:
a. adding water to the vermicompost according to fifth aspect to obtain a mixture,
b. agitating the mixture obtained in step a., and
c. filtering to collect the filtrate,
   wherein the vermicompost extract is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

The seventh aspect of the presently claimed invention is directed to a liquid vermicompost extract, wherein the liquid vermicompost extract is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

The eighth aspect of the presently claimed invention is directed to a process of transferring complex microbiota to an animal comprising
spraying a liquid vermicompost extract onto the animals, preferably to chicklets, ducklings and goslings,
or delivering it to the animals via the drinking water,
wherein the liquid vermicompost extract is free of pathogens selected from the group consisting of species of Salmonella, species of Campylobacter, Histomonas meleagridis and any combination of two or more of the aforementioned, and/or SPF according to Ph. Eur. 9, Ausgabe, Grundwerk 2017, 9.0/5.020200.

The nineth aspect of the presently claimed invention is directed to the inventive feed composition according to the first and second aspects to obtain young animal(s) having complex microbiota.

The tenth aspect of the presently claimed invention is directed to a feed composition according to the first and second aspects to obtain animal(s) with increased weight.

The eleventh aspects of the presently claimed invention is directed to the specific pathogen free vermicompost according to the fifth aspect for raising animal having complex microbiota.

The twelfth aspects of the presently claimed invention is directed to liquid vermicompost extract according to the seventh aspect to obtain animal having complex microbiota.

The thirteenth aspects of the presently claimed invention is directed to liquid vermicompost extract according to the seventh aspects to obtain animal free of pathogen.
wherein the pathogens are selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

The fourteenth aspects of the presently claimed invention is directed to the feed composition according to the first and second aspects or the specific pathogen free vermicompost according to the fifth aspect or the liquid vermicompost extract according to the seventh aspect to reduce the number or the intensity of the feet lesions in an animal.

### DESCRIPTION OF THE DRAWINGS

FIG. 1 shows that Akkermansia muciniphilia, Lactobacillus coleohominis and Bifidobactenum pseudolongum gut inhabitants increased in the feces of the poultry which are fed with worms-vermicompost-mix over the days 2 to 6 of the fattening period.

Taxonomy

[symbol] k_Bacteria; p_Actinobacteria; c_Actinobacteria; o_Bifidobacteriales; f_Bifidobacteriaceae; g_Bifidobacterium; s_pseudolongum

[symbol] k_Bacteria; p_Firmicutes; c_Bacilli; o_Lactobacillales; f_Lactobacillaceae; g_Lactobacillus; s_coleohomins

[symbol] k_Bacteria; p_Verrucomicrobia; c_Verrucomicrobiae; o_Verrucomicrobiales; f_Verrucomicrobiaceae; g_Akkermansia; s_muciniphila

FIG. 2 shows the frequency of birds with sticky feces attached to their back after feeding experimental diets.

FIG. 3 shows compositional differences between boot-sock samples, with (CON+EW_None) and without EW (CON+_None) feeding. The taxa shown are those significantly different between the two conditions (p value<0.1, Wilcoxon test). The left panel shows taxa under-represented in the EW feeding group, while the right panel shows those promoted by the feeding.

[symbol] Actinobacteria; c_Actinobacteria; o_Bifidobacteriales; f_Bifidobacteriaceae; g_Bifidobacterium; s_pseuc

[symbol] Firmicutes; c_Bacilli; o_Lactobacillales: f_Lactobacillaceae; g_Lactobacillus; s_coleohominis

[symbol] Firmicutes; c_Clostridia; o_Clostridiales; f_Lachnospiraceae; g_Blautia

[symbol] Firmicutes; c_Clostridia; o_Clostridiales; f_Lachnospiraceae; g_Clostridium; s_piliforme

[symbol] Firmicutes; c_Erysipelotrichi; o_Erysipelotrichales; f_Erysipelotrichaceae; g_cc_115; s_

[symbol] Proteobacteria; c_Gammaproteobacteria; o_Enterobacteriates; f_Enterobacteriaceae; g_Citrobacter; s_

[symbol] TM7; c_TM7-3; o_l025; f_Rs-045; g_; s_

[symbol] Verrucomicrobia; c_Verrucomicrobiae; o_Verrucomicrobiales; f_Verrucomicrobiaceae; g_Akkermansia; s_

FIG. 4 shows the movement of the chickens fed with worm and chickens fed without worms. The movement was reordered and evaluated using artificial intelligence (AI) and its shows that the chickens fed with worms has improved movement even weeks after the last earthworm feeding.

FIG. 5 shows the microbiota of different parts of worm, soil and pool (worm+soil).

FIG. 6 shows the overall aftertaste for the chickens fed with the worms against the control.

### DETAILED DESCRIPTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or a, b, c, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

In the presently claimed invention the “wet weight” is defined as the composition comprising water content in the range of 5 to 95 wt. % based on overall weight of the composition, preferably the “wet weight” is defined as the composition comprising water content in the range of 10 to 95 wt. % based on overall weight of the composition, more preferably the “wet weight” is defined as the composition comprising water content in the range of 15 to 95 wt. % based on overall weight of the composition, even more preferably the “wet weight” is defined as the composition comprising water content in the range of 20 to 95 wt. % based on overall weight of the composition, most preferably the “wet weight” is defined as the composition comprising water content in the range of 20 to 90 wt. % based on overall weight of the composition, and in particular the “wet weight” is defined as the composition comprising water content in the range of 25 to 90 wt. % based on overall weight of the composition.

In the presently claimed invention the “worm food free of pathogen” is defined as the food is free of pathogens selected from the group consisting of species of Salmonella, species of Campylobacter, Histomonas meleagridis and any combination of two or more of the aforementioned, more preferably the “worm food free of pathogen” is defined as the food is free of pathogens selected from the group consisting of species of Salmonella, species of Campylobacter, Histomonas meleagridis and any combination of two or more of the aforementioned and/or SPF according to Ph. Eur. 9, Ausgabe, Grundwerk 2017, 9.0/5.02.0200.

In the presently claimed invention the “vermicompost free of pathogen” is defined as the vermicompost is free of pathogens selected from the group consisting of species of Salmonella, species of Campylobacter, Histomonas meleagridis and any combination of two or more of the aforementioned, more preferably the “vermicompost free of pathogen” is defined as the vermicompost is free of pathogens selected from the group consisting of species of Salmonella, species of Campylobacter, Histomonas meleagridis and any combination of two or more of the aforementioned and/or SPF according to Ph. Eur. 9, Ausgabe, Grundwerk 2017, 9.0/5.020200.

In the presently claimed invention the “positive microbiota” is defined as the microbes helpful for the animal. It means coexistence of microbes in the gut of the animal(s).

In a first embodiment, the presently claimed invention is directed to a feed composition comprising
at least one living worm,
wherein the worm is free of pathogens selected from the group consisting of species of Salmonella, species of Campylobacter, Histomonas meleagridis and any combination of two or more of the aforementioned.

In another preferred embodiment, the presently claimed invention is directed to a feed composition for young chicken comprising
at least one living worm,
wherein the worm is free of pathogens selected from the group consisting of species of Salmonella, species of Campylobacter, Histomonas meleagridis and any combination of two or more of the aforementioned and/or SPF according to Ph. Eur. 9, Ausgabe, Grundwerk 2017, 90/5020200.

A sample of living worms is regarded as free of species of Salmonella if from 100 g sample of living worms, 25 gram are analyzed according to DIN EN ISO 6579-1, 2017-07 and no species of Salmonella are detected in the analyzed sample.

A sample of living worms is regarded as free of species campylobacter if from an 100 g sample of livings worms, 25 gram are analyzed according to DIN ISO 21528-2, 2017-09 and the total amount of species of campylobacter detected in that sample is 1000000 KbE/g or less.

A sample of living worms is regarded as free of histomonas meleagridis if from 100 g sample of livings worms, 25 gram are analyzed by PCR 18 S and no histomonas meleagridis are detected in the analyzed sample. Sample preparation and analysis are known to the person skilled in the art.

According to presently claimed invention the Specific Pathogen Free (SPF) is the science of developing and maintaining a species of animals free of infection by specified pathogenic micro-organisms. A specified pathogen is any micro-organism for which infection of poultry can produce a recognized disease condition. Specified pathogen free can only be applied to poultry which return negative results in accepted surveillance tests for the-presence of one or more specific pathogens. Testing must be repeated at regular, defined intervals for the SPF status of a flock to remain current. The most SPF flocks will have a limited resident gut microbial flora e.g. species of Salmonella, species of Campylobacter, Histomonas meleagridis, Streptococcus, coliforms, bacterioides, Clostridia spp., even after several SPF generations in isolators with wire floors. An SPF flock comprises a group of birds which share a common environment whether pen, building or isolator. means the animals/worms are free of specific pathogen which causes diseases.

In another preferred embod/ment, the SPF chicken means, the chicken that is free of specific pathogens accord/ng to Ph. Eur. 9, Ausgabe, Grundwerk 2017, 9.0/5.020200.

In another preferred embodiment the worm is an earthworm.

In another preferred embodiment, the earthworm species is selected from the group consisting of Dendrobena veneta, Lumbricus terrestris, Eisenia fetida, Eudrilus eugeniae, Perionyx excavatus and Eisenia andreii more preferably the earth worm species is selected from the group consisting of Dendrobena veneta, Eisenia andrei and Eisenia fetida and most preferably the earth worm species is Dendrobena veneta.

Worms free of pathogens selected from the group consisting of species of Salmonella, species of Campylobacter, Histomonas meleagridis and any combination of two or more of the aforementioned are produced by collecting eggs from worms free of pathogens selected from the group consisting of species of Salmonella, species of Campylobacter, Histomonas meleagridis and any combination of two or more of the aforementioned. Firstly, the eggs are collected, and the mother worm is analyzed for the presence of the specific pathogens. Once it has been confirmed that the mother worm is free of the specific pathogens the eggs are hatched and reared in a pathogen free environment.

SPF worms are produced by collecting eggs from SPF worms. Firstly, the eggs are collected, and the mother worm is analyzed for the presence of the specific pathogens. Once it has been confirmed that the mother worm is free of the specific pathogens the eggs are hatched and reared in a pathogen free environment.

In another preferred embodiment the at least two living worm has an average length in the range of 3.5 cm to 8 cm, and a minimum length of 2 cm and a maximum length of 12 cm; more preferably the at least two living worm has an average length in the range of 4 cm to 7 cm, and a minimum length of 3 cm and a maximum length of 11 cm, even more preferably the at least two living worm has an average length in the range of 4 cm to 7 cm, and a minimum length of 3 cm and a maximum length of 10 cm, most preferably the at least two living worm has an average length in the range of 5 cm to 7 cm, and a minimum length of 4 cm and a maximum length of 10 cm, and in particular the at least two living worm has an average length in the range of 5 cm to 7 cm, and a minimum length of 4 cm and a maximum length of 9 cm.

In another preferred embodiment the at least one living worm in the composition is present in an amount in the range of 40 wt. % to 60 wt. %, preferably 40 to 55 wt.%, more preferably 45 to 55 wt. %, and particularly 50 wt.%, based on the total wet weight of the composition.

In another preferred embodiment the feed composition comprises at least one living worm, wherein the worm is free of pathogens selected from the group consisting of species of Salmonella, species of Campylobacter, Histomonas meleagridis and any combination of two or more of the aforementioned, and the feed composition further comprises vermicompost which is also free of pathogens selected from the group consisting of species of Salmonella, species of Campylobacter, Histomonas meleagridis and any combination of two or more of the aforementioned.

In another preferred embodiment the feed composition for young chicken comprises at least one living worm,
wherein the worm is free of pathogens selected from the group consisting of species of Salmonella, species of Campylobacter, Histomonas meleagridis and any combination of two or more of the aforementioned, and/or SPF according to according to Ph. Eur. 9, Ausgabe, Grundwerk 2017, 9.0/5020200 and the feed composition further comprises vermicompost which is also free of pathogens selected from the group consisting of species of Salmonella, species of Campylobacter, Histomonas meleagridis and any combination of two or more of the aforementioned.

It is preferred that vermicompost and the living worm(s) are free of the same pathogens.

A sample of vermicompost is regarded as free of species of Salmonella if from 100 g sample of vermicompost, 25 gram are analyzed according to DIN EN ISO 6579-1, 2017-07 and no species of Salmonella are detected in the analyzed sample.

A sample of vermicompost is regarded as free of species Campylobacter if from 100 g sample of vermicompost, 25 gram are analyzed according to DIN ISO 21528-2, 2017-09 and the total amount of species of campylobacter detected in that sample is 1000000 KbE/g or less.

A sample of vermicompost is regarded as free of Histomonas meleagridis if from 100 g sample of vermicompost, 25 gram are analyzed by PCR 18S and no Histomonas meleagridis are detected in the analyzed sample. Sample preparation and analysis are known to the person skilled in the art.

Vermicompost is the product of the decomposition process using various species of worms, especially earthworms, to create a mixture of decomposing vegetable or food waste, bedding materials, and vermicast. Vermicast is the end-prodcut of the breakdown of organic matter by worms, especially earthworms.

This process is called vermicomposting, while the rearing of worms for this purpose is called vermiculture.

In another preferred embodiment the vermicompost in the feed composition is present in an amount in the range of 40 wt. % to 60 wt. %, preferably 40 to 55 wt.%, more preferably 45 to 55 wt. %, and particularly 50 wt.%, based on the total wet weight of the composition.

In another preferred embodiment the presently claimed invention is directed to a feed composition comprising
at least one living worm,
wherein the worm is free of pathogens selected from the group consisting of species of Salmonella, species of Campylobacter, Histomonas meleagridis and any combination of two or more of the aforementioned; and
vermicompost,
wherein the at least one living worm in the composition is present in an amount in the range of 40 wt. % to 60 wt. %, and the vermicompost is present in an amount in the range of 40 wt. % to 60 wt. %, based on the total wet weight of the composition.

In another preferred embodiment the presently claimed invention is directed to a feed composition comprising
at least one living worm,
wherein the worm is free of pathogens selected from the group consisting of species of Salmonella, species of Campylobacter, Histomonas meleagridis and any combination of two or more of the aforementioned; and
vermicompost,
wherein the at least one living worm in the composition is present in an amount in the range of 5 wt. % to 40 wt. %, and the vermicompost is present in an amount in the range of 60 wt. % to 95 wt. %, based on the total dry weight of the composition.

In another more preferred embodiment the feed composition comprises at least one living worm,
wherein the worm is free of pathogens selected from the group consisting of species of Salmonella, species of Campylobacter, Histomonas meleagridis and any combination of two or more of the aforementioned; and
vermicompost,
wherein the at least one living worm in the composition is present in an amount in the range of 5 wt. % to 35 wt. %, and the vermicompost is present in an amount in the range of 65 wt. % to 95 wt. %, based on the total dry weight of the composition.

In an even more preferred embodiment the feed composition comprises
at least one living worm,
wherein the worm is free of pathogens selected from the group consisting of species of Salmonella, species of Campylobacter, Histomonas meleagridis and any combination of two or more of the aforementioned; and
vermicompost,
wherein the at least one living worm in the composition is present in an amount in the range of 5 wt. % to 30 wt. %, and the vermicompost is present in an amount in the range of 70 wt. % to 95 wt. %, based on the total dry weight of the composition

In an even more preferred embodiment the feed composition comprises
at least one living worm,
wherein the worm is free of pathogens selected from the group consisting of species of Salmonella, species of Campylobacter, Histomonas meleagridis and any combination of two or more of the aforementioned; and
vermicompost,
wherein the at least one living worm in the composition is present in an amount in the range of 5 wt. % to 25 wt. %, and the vermicompost is present in an amount in the range of 75 wt. % to 95 wt. %, based on the total dry weight of the composition.

In particularly preferred embodiment the feed composition comprises
at least one living worm,
wherein the worm is free of pathogens selected from the group consisting of species of Salmonella, species of Campylobacter, Histomonas meleagridis and any combination of two or more of the aforementioned; and
vermicompost,
wherein the at least one living worm in the composition is present in an amount in the range of 5 wt. % to 20 wt. %, and the vermicompost is present in an amount in the range of 80 wt. % to 95 wt. %, based on the total dry weight of the composition.

In another preferred embodiment the feed composition further comprises worm food.

In another preferred embodiment the feed composition further comprises the worm food in an amount in the range of 1 wt. % to 50 wt. %, based on the total wet weight of the composition.

In another preferred embodiment the presently claimed invention is directed to a feed
composition comprising
at least one living worm,
vermicompost, and
worm food
wherein the worm is free of pathogens selected from the group consisting of species of Salmonella, species of Campylobacter, Histomonas meleagridis and any combination of two or more of the aforementioned;
the at least one living worm in the composition is present in an amount in the range of 30 wt. % to 60 wt. %, the vermicompost is present in an amount in the range of 30 wt. % to 60 wt. 35%, and the worm food is present in an amount in the range of 1 wt. % to 40 wt. %, based on the total wet weight of the composition.

In another preferred embodiment the presently claimed invention is directed to a feed composition comprising
at lest one living worm,
vermicompost, and
worm food
wherein the worm is free of pathogens selected from the group consisting of species of Salmonella, species of Campylobacter, Histomonas meleagridis and any combination of two or more of the aforementioned;
the at least one living worm in the composition is present in an amount in the range of 1 wt. % to 40 wt. %, the vermicompost is present in an amount in the range of 30 wt. % to 80 wt. %, and the worm food present in an amount in the range of 1 wt. % to 69 wt. %, based on the total dry weight of the composition.

More preferably the feed composition comprises
at least one living worm,
vermicompost, and
worm food
wherein the worm is free of pathogens selected from the group consisting of species of Salmonella, species of Campylobacter, Histomonas meleagridis and any combination of two or more of the aforementioned;
the at least one living worm in the composition is present in an amount in the range of 1 wt. % to 30 wt. %, the vermicompost is present in an amount in the range of 30 wt. % to 70 wt. %, and the worm food present in an amount in the range of 10 wt. % to 69 wt. %, based on the total dry weight of the composition.

Even more preferably the feed composition comprises
at least one living worm,
vermicompost, and
worm food
wherein the worm is free of pathogens selected from the group consisting of species of Salmonella, species of Campylobacter, Histomonas meleagridis and any combination of two or more of the aforementioned;
the at least one living worm in the composition is present in an amount in the range of 1 wt. % to 25 wt. %, the vermicompost is present in an amount in the range of 30 wt. % to 70 wt.

%, and the worm food present in an amount in the range of 10 wt. % to 50 wt. %, based on the total dry weight of the composition;

Even more preferably the feed composition comprises
at least one living worm,
vermicompost, and
worm food
wherein the worm is free of pathogens selected from the group consisting of species of Salmonella, species of Campylobacter, Histomonas meleagridis and any combination of two or more of the aforementioned;
the at least one living worm in the composition is present in an amount in the range of 3 wt. % to 20 wt. %, the vermicompost is present in an amount in the range of 40 wt. % to 70 wt. %, and the worm food present in an amount in the range of 10 wt. % to 50 wt. %, based on the total dry weight of the composition;

In particular the feed composition comprises
at least one living worm,
vermicompost, and
worm food
wherein the worm is free of pathogens selected from the group consisting of species of Salmonella, species of Campylobacter, Histomonas meleagridis and any combination of two or more of the aforementioned;
the at least one living worm in the composition is present in an amount in the range of 3 wt. % to 15 wt. %, the vermicompost is present in an amount in the range of 40 wt. % to 60 wt. %, and the worm food present in an amount in the range of 20 wt. % to 40 wt. %, based on the total dry weight of the composition.

In another preferred embodiment, the feed composition comprises
at least one living worm, and
at least one standard feed for an animal,
wherein the worm is free of pathogens selected from the group consisting of species of Salmonella, species of Campylobacter, Histomonas meleagridis and any combination of two or more of the aforementioned.

In another preferred embodiment, the feed composition for young chicken comprises
at least one living worm, and
at least one standard feed for an animal,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 90/502.0200.

The term standard feed refers to the feed which is normally feed to specific animals. Based on his knowledge, the person skilled in the art can easily chose and pick the respective standard feed(s) for specific animals. Preferably, the standard feed comprises corn and/orsoybean More preferably the standard feed is corn, soybean or a mixture of corn and soybean. In the presently claimed invention the “standard feed is free of pathogen” is defined as the standard feed is free of pathogens selected from the group consisting of species of Salmonella, species of Campylobacter, Histomonas meleagridis and any combination of two or more of the aforementioned, more preferably the “standard feed free of pathogen” is defined as the standard feed is free of pathogens selected from the group consisting of species of Salmonella, species of Campylobacter, Histomonas meleagridis and any combination of two or more of the aforementioned and/or SPF according to Ph. Eur. 9, Ausgabe, Grundwerk 2017, 9.0/5.020200.

In another preferred embodiment, the feed composition comprises
at least one living worm,
vermicompost, and
standard feed for an animal wherein animals selected from the group consisting of young chicklet, piglet, lambs, calves, duckling, and gosling,
wherein the worm is free of pathogens selected from the group consisting of species of Salmonella, species of Campylobacter, Histomonas meleagridis and any combination of two or more of the aforementioned.

In another preferred embodiment, the feed composition for young chicken comprises
at least one living worm,
vermicompost, and
standard feed for the young chicken,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

In another preferred embodiment, the feed composition for young chicken comprises
at least one living worm,
vermicompost,
worm food, and
standard feed for the young chicken,
wherein the worm is free of pathogens selected from the group consisting of species of Salmonella, species of Campylobacter, Histomonas meleagridis and any combination of two or more of the aforementioned.

In another preferred embodiment, the feed composition for young chicken comprises
at least one living worm,
vermicompost,
worm food, and
standard feed for the young chicken,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200.

In another preferred embodiment, the feed composition for young chicken comprises
at least one living worm, and
standard feed for the young chicken,
wherein the worm is free of pathogens selected from the group consisting of species of Salmonella, species of Campylobacter, Histomonas meleagridis and any combination of two or more of the aforementioned, and
the at least one living worm in the composition is present in an amount in the range of 0.05 wt. % to 1.0 wt. %, and the standard feed for the animal is present in an amount in the range of 99 wt. % to 99.95 wt. %, based total dry weight of the composition.

In another preferred embodiment, the feed composition for young chicken comprises
at least one living worm, and
standard feed for the young chicken,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 90/5020200; and
the at least one living worm in the composition is present in an amount in the range of 0.05 wt. % to 1.0 wt. %, and the standard feed for the animal is present in an amount in the range of 99 wt. % to 99.95 wt. %, based total dry weight of the composition.

In another preferred embodiment, the feed composition for young chicken comprises
at least one living worm,
vermicompost, and
standard feed for the young chicken,
wherein the worm is free of pathogens selected from the group consisting of species of Salmonella, species of Campylobacter, Histomonas meleagridis and any combination of two or more of the aforementioned, and
the at least one living worm in the composition is present in an amount in the range of 0.02 wt. % to 1.0 wt. %, the vermicompost is present in an amount in the range of 0.05 wt. % to 2.0 wt. %, and the standard feed for the animal is present in an amount in the range of 97 wt. % to 99.93 wt. %, based total dry weight of the composition.

In another preferred embodiment, the feed composition for young chicken comprises
at least one living worm,
vermicompost, and
standard feed for the young chicken,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.020200; and
the at least one living worm in the composition is present in an amount in the range of 0.02 wt. % to 1.0 wt. %, the vermicompost is present in an amount in the range of 0.05 wt. % to 2.0 wt. %, and the standard feed for the animal is present in an amount in the range of 97 wt. % to 99.93 wt. %, based total dry weight of the composition.

In another preferred embodiment, the feed composition for young chicken comprises
at least one living worm,
vermicompost,
worm food, and
standard feed for the young chicken,
wherein the worm is free of pathogens selected from the group consisting of species of Salmonella, species of Campylobacter, Histomonas meleagridis and any combination of two or more of the aforementioned, and
the at least one living worm in the composition is present in an amount in the range of 0.02 wt. % to 1.0 wt. %, the vermicompost is present in an amount in the range of 0.05 wt. % to 2.0 wt. %, worm food in the composition is present in an amount in the range of 0.02 wt. % to 1.0 wt. %,and the standard feed for the animal is present in an amount in the range of 96 wt. % to 99.91 wt. %, based total dry weight of the composition.

In another preferred embodiment, the feed composition for young chicken comprises
at least one living worm,
vermicompost,
worm food, and
standard feed for the young chicken,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 90/5020200; and
the at least one living worm in the composition is present in an amount in the range of 0.02 wt. % to 1.0 wt. %, the vermicompost is present in an amount in the range of 0.05 wt. % to 2.0 wt. %, worm food in the composition is present in an amount in the range of 0.02 wt. % to 1.0 wt. %,and the standard feed for the animal is present in an amount in the range of 96 wt. % to 99.91 wt. %, based total dry weight of the composition.

In another preferred embodiment, the worm food throughout the specification is free of pathogens selected from the group consisting of species of Salmonella, species of Campylobacter, Histomonas meleagridis and any combination of two or more of the aforementioned.

Within the context of the presently claimed invention the worm foods are selected from, but not limited to, peat, straw, hay, ground cereals, and limestone.

A sample of worm food is regarded as free of species of Salmonella if from 100 g sample of worm food, 25 grams are analyzed according to DIN EN ISO 6579-1, 2017-07 and no species of salmonella are detected in the analyzed sample.

A sample of worm food is regarded as free of species Campylobacter if from 100 g sample of worm food, 25 gram are analyzed according to DIN ISO 10272-1: 2017-09 and the total amount of species of campylobacter detected in that sample is 0 CFU/g.

A sample of worm food is regarded as free of Histomonas meleagridis if from 100g sample of worm food, 25 grams are analyzed by PCR 18S and no Histomonas meleagridis are detected in the analyzed sample. Sample preparation and analysis are known to the person skilled in the art.

In another embodiment, the presently claimed invention is directed to a process of feeding animals comprising feeding the animal with any of compositions as described above in an amount in the range of 1 mg to 50 mg living worm/g body weight of the animal per day.

In another preferred embodiment, the animal is selected from the group consisting of chicklet, piglet, lambs, calves, duckling, and gosling, more preferably the animal is selected from the group consisting of chicklets, piglet, lambs, duckling, and gosling, even more preferably the animal is selected from the group consisting of chicklets, piglet, duckling, and gosling, most preferably the animal is selected from the group consisting of chicklets, duckling, and gosling, and in particular the animal is chicklets.

In another preferred embodiment, the chicklet, the duckling and the gosling is given/fed 2 mg to 10 mg, more preferably the chicklet, the duckling and the gosling is given/fed 2 mg to 8 mg, most preferably the chicklet, the duckling and the gosling is given/fed 4 mg to 8 mg, and in particular preferably the chicklet, the duckling and the gosling is given/fed 5 mg to 7 mg, of feed composition per g animal weight per day.

In another preferred embodiment, the chicklet, the duckling and the gosling is given/fed 200 mg to 300 mg, more preferably the chicklet, the duckling and the gosling is given/fed 200 mg to 280 mg, most preferably the chicklet, the duckling and the gosling is given/fed 220 mg to 280 mg, and in particular preferably the chicklet, the duckling and the gosling is given/fed 220 mg to 260 mg, of feed composition per chicklet or duckling or gosling per day.

In another preferred embodiment, the piglet is given 1 to 10 g, preferably 2 to 8 g, more preferably 3 to 8 g, most preferably 4 to 8 g, and in particular 5 to 7 g of feed composition per kg piglet weight per day.

In another preferred embodiment, the lamb is given 1 to 10 g, preferably 2 to 8 g, more preferably 3 to 8 g, most preferably 4 to 8 g, and in particular 5 to 7 g of feed composition per kg lamb weight per day.

In another preferred embodiment, the calf is given 1 to 10 g, preferably 2 to 8 g, more preferably 3 to 8 g, most preferably 4 to 8 g, and in particular 5 to 7 g of feed composition per kg calf weight per day.

In another preferred embodiment, the animal or young one, also referred to as young animal(s), is having age in the range of 1 to 10 days, more preferably the animal is having age in the range of 1 to 9 days, most preferably the animal is having age in the range of 1 to 8 days, and in particular the animal is having age in the range of 1 to 7 days.

In another preferred embodiment, the animal is given/fed the composition one time per day.

In another embodiment the presently claimed invention is directed to a method of producing vermicompost comprising the steps of:
a. raising worms from pathogen free breeding stock;
b. feeding the worms with pathogen free feed;
c. keeping/raising the worms in pathogen free surrounding; and
d. collecting the vermicompost,
   wherein “pathogen free” is free of pathogens selected from the group consisting of species of Salmonella, species of Campylobacter, Histomonas meleagndis and any combination of two or more of the aforementioned.

In another preferred embodiment the pathogen free breeding stock is defined as the parent stock that is free of pathogen selected from the group consisting of species of Salmonella, species of Campylobacter, Histomonas meleagndis and any combination of two or more of the aforementioned.

In another preferred embodiment the pathogen free feed for worm is defined as the feed that is free of pathogens selected from the group consisting of species of Salmonella, species of Campylobacter, Histomonas meleagndis and any combination of two or more of the aforementioned.

In another preferred embodiment the pathogen free surrounding is defined as the surrounding of the worms where these are raised which is free of pathogens selected from the group consisting of species of Salmonella, species of Campylobacter, Histomonas meleagndis and any combination of two or more of the aforementioned.

In another embodiment, the presently claimed invention is directed to a pathogen free vermicompost, wherein “free of pathogen” is free of pathogens selected from the group consisting of species of Salmonella, species of Campylobacter, Histomonas meleagridis and any combination of two or more of the aforementioned.

The species of Salmonella is determined in accordance with DIN EN ISO 6579-1; 2017-07.

The species of Campylobacter is determined in accordance with DIN EN ISO 10272-1, September; 2017

The Histomonas meleagridis is determined in accordance with Real time PCR 18S.

In another preferred embodiment, the pathogen free vermicompost further comprises the worm food.

In another preferred embodiment the vermicompost comprises the worm food in an amount in the range of 1 wt. % to 50 wt. %, based on the total wet weight of the vermicompost.

In another embodiment, the presently claimed invention is directed to a process for preparing a liquid vermicompost extract comprising the steps of:
a. adding water to the vermicompost to obtain a mixture,
b. agitating the mixture obtained in step a. for a period, and
c. filtering to collect the filtrate.

In another preferred embodiment, in step a. of the process the vermicompost is preferably provided in a bag.

In another preferred embodiment, the ratio of water to the vermicompost dilution in step a. is in the range of 3:1 to 200:1, more preferably the ratio of water to the vermicompost dilution in step a. is in the range of 3:1 to 100:1, even more preferably the ratio of water to the vermicompost dilution in step a. is in the range of 3:1 to 50:1, most preferably the ratio of water to the vermicompost dilution in step a. is in the range of 3:1 to 25:1, and in particular the ratio of water to the vermicompost dilution in step a. is in the range of 3:1 to 10:1, wherein each case is by weight.

In another preferred embodiment the filtering in step c. is performed using a sieve with a mesh size of 40 μm to 3000 μm, more preferably the filtering in step c. is performed using a sieve with a mesh size of 40 μm to 1000 μm, even more preferably the filtering in step c. is performed using a sieve with a mesh size of 80 μm to 5000 μm, most preferably the filtering in step c. is performed using a sieve with a mesh size of 100 μm to 300 μm; and in particular the filtering in step c. is performed using a sieve of with a mesh size of 100 μm to 1500 μm.

In another preferred embodiment, the bag is preferably made of cotton.

In another preferred embodiment, the vermicompost is filled in a water permeable bag.

In another preferred embodiment, the vermicompost comprised in the bag is agitated for a period of 2 minutes to 1 hour, preferably the vermicompost comprised in the bag is agitated for a period of 2 minutes to 45 minutes, more preferably the vermicompost comprised in the bag is agitated for a period of 5 minutes to 30 minutes, even more preferably the vermicompost comprised in the bag is agitated for a period of 5 minutes to 15 minutes, and in particular the bag comprising the vermicompost is agitated for a period of 5 minutes to 10 minutes.

In another preferred embodiment, the step c, comprises a step of filtering the vermicompost mixture to obtain a filtrate comprising the vermicompost extract.

In another preferred embodiment, the step c, comprises removing the bag to obtain the vermicompost extract.

In another preferred embodiment, the water in step a) is free of chlorine and having temperature in the range of 5-20° C., more preferably 5 to 15° C., most preferably 8 to 15° C., and in particular preferably 10-12° C. The chlorine content in the water determined in accordance to ISO 7393-2:2017.

In another embodiment, the presently claimed invention is directed to a liquid vermicompost extract obtained according to the process as defined above.

In another embodiment, the presently claimed invention is directed to a process of transferring complex microbiota to at least one animal comprising spraying the liquid vermicompost extract onto the at least one animal.

In another preferred embodiment, the animal is selected from the group consisting of chicklets, ducklings and goslings.

In another embodiment, the presently claimed invention is directed to a process of transferring complex microbiota to an animal via the drinking water.

In another preferred embodiment, the animals are selected from the group consisting of chicklets, ducklings, piglet, lamb, calf and goslings.

In another preferred embodiment, the liquid vermicompost extract is diluted with water in a ratio of 1.0:0.5 to 1.0:2.0 before spraying onto the at least one animal; more preferably the liquid vermicompost extract is diluted with water in a ratio of 1.0:0.8 to 1.0:1.5 before spraying onto the animals, and most preferably the liquid vermicompost extract is diluted with water in a ratio of 1.0:1.0: before spraying onto the at least one animal.

In another preferred embodiment, the amount of liquid sprayed onto the at least one animal is from 0.1 to 5 ml per animal, preferably the amount of liquid sprayed onto the at least one animal is from 0.1 to 3 ml per animal, even more preferably the amount of liquid sprayed onto the at least one animal is from 0.1 to 2 ml per animal, most preferably the amount of liquid sprayed onto the at least one animal is from 0.1 to 1.0 ml per animaland in particular the amount of liquid sprayed onto the at least one animal is from 0.2 to 0.4 ml per animal.

In another embodiment, the presently claimed invention is directed to the feed composition as described above to obtain young animal having complex microbiota characterized via 16S bacterial community analysis. The method includes isolation of DNA from chicken digesta or feces followed by 16S rRNA gene amplification and NGS sequencing.

Within the context of the presently claimed invention the DNA-extraction prior to ilumna sequencing is performed according to a method described in PLoS ONE 13(8):e0202858. https://doi.org/10.1371/journal.pone.0202858.

In another embodiment, the presently claimed invention is directed to a feed composition as described above to obtain animal with increased weight.

In another embodiment, the presently claimed invention is directed to specific pathogen free vermicompost as described above for raising animal having complex microbiota characterized via standard PCR testing. The complex microbiota is characterized via 16S bacterial community analysis. The method includes isolation of DNA from chicken digesta or feces followed by 16S rRNA gene amplification and NGS sequencing.

In another embodiment, the presently claimed invention is directed to the liquid vermicompost extract as described above to obtain animal having complex microbiota characterized via standard PCR testing. The complex microbiota is characterized via 16S bacterial community analysis. The method includes isolation of DNA from chicken digesta or feces followed by 16S rRNA gene amplification and NGS sequencing.

In another embodiment, the presently claimed invention is directed to the liquid vermicompost extract as described above to obtain animal having higher counts of gut bacteria like Akkermansia muciniphilia, Lactobacillus coleohominis and Bifidobacter pseudolongo in their feces via standard PCR testing. The complex microbiota is characterized via 16S bacterial community analysis. The method includes isolation of DNA from chicken digesta or feces followed by 16S rRNA gene amplification and NGS sequencing.

In another embodiment, the presently claimed invention is directed to specific pathogen free vermicompost for raising animal having higher counts of gut bacteria like Akkermansia muciniphilia, Lactobacillus coleohominis and Bifidobacter pseudolongo in their feces characterized via standard PCR testing. The complex microbiota is characterized via 16S bacterial community analysis. The method includes isolation of DNA from chicken digesta or feces followed by 16S rRNA gene amplification and NGS sequencing.

In another embodiment, the presently claimed invention is directed to the of liquid vermicompost extract to obtain animal having higher counts of gut bacteria like Akkermansia muciniphilia, Lactobacillus coleohominis and Bifidobacter pseudolongo in their feces characterized via standard PCR testing. The complex microbiota is characterized via 16S bacterial community analysis. The method includes isolation of DNA from chicken digesta or feces followed by 16S rRNA gene amplification and NGS sequencing.

In another preferred embodiment the term “higher” means increase in the counts of any one of the or any two of the or all of the bacteria like Akkermansia muciniphilia, Lactobacillus coleohominis and Bifidobacter pseudolongo at least by 10%, preferably at least by 20%, more preferably by 40%, even more preferably by 80%, most preferably by 100% and in particular by 100%, in each case compared to animals that not fed with inventive composition. To measure this the samples were collected from feces.

In another embodiment, the presently claimed invention is directed to liquid vermicompost extract as described above to obtain animal free of pathogen,
wherein the pathogens are selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 90/5020200.

In another embodiment, the presently claimed invention is directed to the feed composition as described above or the specific pathogen free vermicompost as described above or the liquid vermicompost extract as described above to reduce the number or the intensity of the feet lesions in an animal.

In the following, there is provided a list of embodiments to further illustrate the present disclosure without intending to limit the disclosure to the specific embodiments listed below.
1. A feed composition comprising
at least one living worm,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5020200.
2. The feed composition according to embodiment 1, wherein the composition comprises at least two living worms having an average length in the range of 3.5 cm to 8 cm, and a minimum length of 2 cm and a maximum length of 12 cm.
3. The feed composition according to any one of the embodiments 1 to 2, wherein the at least one living worm in the composition is present in an amount in the range of 40 wt. % to 60 wt. %, preferably 45 to 55 wt.%, particularly 50 wt.%, based on the total wet weight of the composition.
4. The feed composition according to any one of the embodiments 1 to 3, wherein the composition further comprises vermicompost.
5. The composition according to embodiment 4, wherein the vermicompost is present in an amount in the range of 40 wt. % to 60 wt. %, preferably 45 to 55 wt.%, particularly 50 wt.%, based on the total wet weight of the composition.
6. A feed composition comprising:
   a. vermicompost in an amount in the range of 0,05 wt. % to 2,0 wt. % based on the total dry weight of the composition; and
   b. worms in an amount in the range of 0,02′ wt. % to 1,0′ wt. % based on the total dry weight of the composition.
7. A process for feeding animals for non-therapeutic use comprising feeding the animal with a composition according to embodiments 1 to 6 in an amount in the range of 2 mg to 50 mg living worm/g body weight of the animal per day.
8. The process according to embodiment 7, wherein the animal is selected from the group consisting of chicklet, piglet, lambs, calves, duckling, and gosling, preferably chicklets.
9. The process according to embodiment 8, wherein the chicklet, the duckling and the gosling is given 200 mg to 300 mg, preferably 200 to 250 mg, of feed composition per g animal weight per day.
10. The process according to embodiment 8, wherein the piglet is given 1 to 6 g, preferably 2 to 5 g, of feed composition per kg animal weight per day.
11. The process according to embodiment 8, wherein the lamb is given 1 to 6 g, preferably 2 to 5 g of feed composition per kg animal weight per day.
12. The process according to embodiment 8, wherein the calf is given 1 to 10 g, preferably 2 to 8 g, of feed composition per kg animal weight per day.
13. The process according to any one of the embodiments 7 to 13, wherein the animal is having age in the range of 1 to 500 days, more preferably the chicklet, duckling, gosling having age in the range of 1 to 10 days, most preferably the chicklet, duckling, gosling having age in the range of 1 to 7 days.
14. The process according to any one of the embodiments 7 to 13, wherein the animal is feed the composition one time per day.
15. A method of producing vermicompost comprising the steps of:
   a. raising worms from pathogen free breeding stock;
   b. feeding the worms with pathogen free feed;
   c. keeping/raising the worms in pathogen free surrounding; and
   d. collecting the vermicompost,
wherein” pathogen free” is free of pathogens selected from the group consisting of species of Salmonella, species of Campylobacter, Histomonas meleagridis and any combination of two or more of the aforementioned.
16. The pathogen free vermicompost obtained according to embodiment 15.
17. A process for preparing a liquid vermicompost extract comprising the steps of:
   a. adding water to the vermicompost obtained according to embodiment 16 to obtain a mixture,
   b. agitating the mixture obtained in step a. for a period of 1 minutes to 1 hour, and
   c. filtering to collect the filtrate.
18. The process according to embodiment 17, wherein the water is free of chlorine and having temperature in the range of 5-20° C., preferably 11-12° C.
19. A liquid Vermicompost extract obtained according to any one of the embodiments 17 to 18.

20. Process of transferring complex microbiota to an animal comprising
spraying the liquid vermicompost extract according to embodiment 19 onto the animals, preferably to chicklets, ducklings and goslings,
or delivering to the animals via the drinking water.
21. The process according to embodiment 20, wherein the liquid vermicompost extract according to embodiment 19 is optionally diluted with water in a ratio of 1.0:0.5 to 1.0: 2.0 before spraying onto the animals.
22. The process according to embodiment 20, wherein the amount of liquid sprayed onto the animals is from 0.1 mL to 5 mL per animal.
23. A feed composition according to any one of the embodiments 1 to 6 to obtain young animal having higher counts of gut bacteria like Akkermansia muciniphilia, Lactobacillus coleohominis and Bifidobacter pseudolongo in their feces via standard PCR testing. The complex microbiota is characterized via 16S bacterial community analysis. The method includes isolation of DNA from chicken digesta or feces followed by 16S rRNA gene amplification and NGS sequencing.
24. The feed composition according to anyone of the embodiments 1 to 6 to obtain animal with increased weight.
25. Specific pathogen free vermicompost according to embodiment 16 for raising animal having higher counts of gut bacteria like Akkermansia muciniphilia, Lactobacillus coleohominis and Bifidobacter pseudolongo in their feces characterized via standard PCR testing. The complex microbiota is characterized via 16S bacterial community analysis. The method includes isolation of DNA from chicken digesta or feces followed by 16S rRNA gene amplification and NGS sequencing.
26. The liquid vermicompost extract according to embodiment 19 to obtain animal having higher counts of gut bacteria like Akkermansia muciniphilia, Lactobacillus coleohominis and Bifidobacter pseudolongo in their feces characterized via standard PCR testing. The complex microbiota is characterized via 16S bacterial community analysis. The method includes isolation of DNA from chicken digesta or feces followed by 16S rRNA gene amplification and NGS sequencing.
27. The liquid vermicompost extract according to embodiment 19 to obtain animal free of pathogen,
wherein the pathogens are selected from the group consisting of species of Salmonella, species of Campylobacter, Histomonas meleagridis and any combination of two or more of the aforementioned.
28. A feed composition according to any one of the embodiments 1 to 6 or the specific pathogen free vermicompost according to embodiment 16 or the liquid vermicompost extract according to embodiment 19 to reduce the number or the intensity of the feet (pododermatitis) lesions in an animal.
29. A feed composition according to any one of the embodiments 1 to 6 or the specific pathogen free vermicompost according to embodiment 16 or the liquid vermicompost extract according to embodiment 19 to reduce the number or the intensity of the feet lesions in an animal.

While the presently claimed invention has been described in terms of its specific embodiments, certain modifications and equivalents will be apparent to those skilled in the art and are intended to be included within the scope of the presently claimed invention.

### EXAMPLES

### Animals and Experimental Design

Male broiler birds (Cobb-500) were used in a feeding trial with two identical runs (R) with 240 birds each (N=480). The birds were purchased from a commercial hatchery (Cobb Germany Avimex GmbH, Bruterei Wiesenena, Wiedemar, Germany). The chicks received vaccinations against infectious bronchitis and Newcastle disease after hatching at the hatchery. Per run and starting from the first day (d) of life onwards, the birds were fed either a corn-soybean-meal-based control diet (CON+, n=120) or CON+ supplemented with either 1% (dry matter, DM) earthworms (CON+EW; n=60) or vermicompost (CON+VC; n=60) for 8 d (Period 1; P1). On d 8, half the birds in each group were sampled for digesta and intestinal size measurements. Half the remaining birds on the CON+ diet (n=30 per group) were either kept on the same diet for further 8 d (P2) or given another diet that replaced approximately 50% of corn in the CON+ with wheat, barley and rye to produce a challenge diet with higher non-starch polysaccharides (NSP) (i.e. negative control diet, CON−). The birds consuming EW and VC in P1 were fed the CON− diet in P2 (i.e. CON−EW and CON−VC, respectively). On d 16, the remaining birds were sacrificed. Since feed change was done on d8, birds of respective groups consumed both diets (i.e. diets of P2 were offered first at around noon time on d8. In the morning of d8, all the birds received their respective diets offered in P1). Experimental design and feeding groups in relation to time are summarized in the following Tables 1 and 2.

TABLE 1

| | Period 1(0-7 days) | Period 2(8-15 days) |
|---|---|---|
| Groups | (n = 60) | (n = 30) |
| Group 1 | CON+ | CON+ |
| Group 2 | CON+ | CON− |
| Group 3 | CON + Earthworm | CON− & Earthworm |
| Group 4 | CON + vermicompost | CON− & Vermicompost |

CON+: positive control diet; CON−: negative control diet; CON + EW: positive control diet plus 1% of earthworm; CON + VC: positive control diet supplemented with 1% vermicompost; CON − EW: negative control diet plus 1% earthworm; CON − VC: negative control diet supplemented with 1% vermicompost.

| | Rooms | Pen 1 | Pen 2 | Pen 3 | Pen 4 | Pen 5 | Pen 6 |
|---|---|---|---|---|---|---|---|
| Period 1 | Room 1 | CON + VC | CON + EW | CON+ | CON+ | CON+ | CON+ |
| N = | Room 2 | CON + EW | CON+ | CON + VC | CON+ | CON + EW | CON + VC |
| 10/pen | Room 3 | CON+ | CON + VC | CON + EW | CON + VC | CON+ | CON + EW |
| | Room 4 | CON+ | CON+ | CON+ | CON + EW | CON + VC | CON+ |

| Rooms | Pen 1 | Pen 2 | Pen 3 | Pen 4 | Pen 5 | Pen 6 |
|---|---|---|---|---|---|---|
| Room 1 | CON − VC | CON − EW | CON− | CON− | CON− | CON− |
| Room 2 | CON − EW | CON− | CON − VC | CON− | CON − EW | CON − VC |
| Roo 3m | CON− | CON − VC | CON − EW | CON − VC | CON− | CON − EW |
| Room 4 | CON− | CON− | CON− | CON − EW | CON − VC | CON− |

Allocation of the birds to the experimental diets offered in pens of different rooms with respect to timeline. In each run, birds on each diet were allocated to different rooms and pens, i.e. the above-presented allocation reflects only one of the runs. Number of birds in each pen was n=10 in P1 and n=5 in P2.

Climatic conditions (light, relative humidity) provided in the rooms were in line with recommendations for commercially reared broilers. The climatic conditions were controlled by an automatic system to ensure uniform temperature, light and aeration conditions across the pens within and between rooms. Wood shaving was used as litter material and was placed on the ground of each pen in equal amount to (i.e. 1600 g/pen). Litter moisture content was determined on d8 and d16

### Nutrient and Energy Contents of the Experimental Diets

Composition and calculated approximate nutrient contents of the diets are given in Table 3. All 4 diets had similar energy (ca. 12.4 MJ/kg) and protein (220 g CP/ kg) contents. Feed was manufactured by a company, Research Diet Services BV, Wijk bij Duurstede, The Netherlands. No NSP degrading enzymes were added to the diets. Diets were analyzed at the FBN for their amino acid (AA) composition after acid hydrolysis with HPLC equipped with a fluorescence detector (Series 1200; Agilent Technologies, Waldbronn, Germany) as described by Kuhla et al., 2010. The AA contents in diets were determined quantitatively with liquid ion exchange chromatography using Biochrom 20; Pharmacia LKB Biochrom Ltd., Cambridge, U.K. The acid hydrolysates were prepared according to Hennig et al. (2004). Analyzed AA compositions of the diets and the AA composition of earthworms is given in table 4.

TABLE 3
Composition and calculated nutrient and energy
contents of the experimental diets*

| Ingredients, g/kg | CON+ | CON− | CON + VC | CON − VC |
|---|---|---|---|---|
| Corn | 580.85 | 278.85 | 533.80 | 229.70 |
| Soybean meal | 349.00 | 334.00 | 354.00 | 340.00 |
| (480 CP) | | | | |
| Soybean oil | 25.00 | 41.00 | 40.00 | 57.00 |
| (37.5 MJ) | | | | |
| Wheat | 0.00 | 100.00 | 0.00 | 100.00 |
| Barley | 0.00 | 100.00 | 0.00 | 100.00 |
| Rye | 0.00 | 100.00 | 0.00 | 100.00 |
| Vermicompost | 0.00 | 0.00 | 28.25 | 28.25 |
| Premix1 | 5.00 | 5.00 | 5.00 | 5.00 |
| Lime fine | 14.80 | 14.80 | 13.40 | 13.20 |
| Monocalcium | 14.40 | 14.00 | 14.40 | 14.40 |
| phosphate | | | | |
| NaCl | 1.90 | 1.90 | 1.90 | 1.90 |
| NaHCO3 | 2.80 | 2.80 | 2.80 | 2.80 |
| L-Lysine HCl | 2.00 | 2.45 | 1.95 | 2.40 |
| DL-Methionine | 3.15 | 3.40 | 3.25 | 3.45 |
| L-Threonine | 0.80 | 1.15 | 0.85 | 1.15 |
| L-Valine | 0.30 | 0.65 | 0.40 | 0.75 |

Calculated contents, g/kg

| | | | | |
|---|---|---|---|---|
| Dry matter | 879.7 | 880.7 | 866.9 | 868.1 |
| Ash | 61.2 | 61.5 | 59.6 | 60.1 |
| Crude protein | 220.1 | 220.1 | 220.0 | 220.3 |
| Crude fat | 52.8 | 64.4 | 66.4 | 79.0 |
| Linoleic acid | 26.2 | 30.3 | 33.0 | 37.6 |
| Crude fibre | 21.4 | 25.9 | 22.0 | 26.5 |
| Carbohydrates | 521.1 | 507.9 | 488.3 | 473.8 |
| Starch | 384.7 | 354.6 | 353.9 | 322.4 |
| Sugars | 47.0 | 50.7 | 46.7 | 50.3 |
| NDF | 88.5 | 90.3 | 84.2 | 85.9 |
| ADF | 33.9 | 36.4 | 49.8 | 52.4 |
| Ca | 9.0 | 9.0 | 9.0 | 9.0 |
| P-Total | 6.9 | 6.9 | 6.8 | 6.9 |
| P-Available | 4.4 | 4.4 | 4.4 | 4.4 |
| P-Dig. Pig | 4.0 | 3.9 | 4.0 | 4.0 |
| P-Dig. Poultry | 4.2 | 4.2 | 4.2 | 4.2 |
| Ca/dP Poultry | 2.15 | 2.16 | 2.15 | 2.12 |
| Ca:P | 1.31 | 1.31 | 1.32 | 1.3 |
| Mg | 1.6 | 1.6 | 1.7 | 1.7 |
| K | 9.7 | 9.6 | 9.6 | 9.6 |
| Na | 1.6 | 1.6 | 1.6 | 1.6 |
| Cl | 1.9 | 2.1 | 1.9 | 2.0 |
| Base-excess | 261.2 | 256.5 | 262 | 257.7 |
| (meq/kg) | | | | |

Metabolizable energy (MJ/kg)

| | | | | |
|---|---|---|---|---|
| AMEn German | 12.39 | 12.39 | 12.39 | 12.40 |
| ME Broiler-Dutch | 12.10 | 11.95 | 12.07 | 11.94 |
| ME Layer-Dutch | 12.42 | 12.34 | 12.41 | 12.34 |
| AMEn nutrients | 12.26 | 12.20 | 12.20 | 12.16 |

TABLE 4
Amino acid composition of the experimental diets and
earthworms (mg per g DM of air dried samples).

| Mg/g DM | CON+ | CON− | CON + VC | CON − VC | Earthworm* |
|---|---|---|---|---|---|
| Asp/Asn | 22.7 | 22.9 | 23.0 | 22.4 | 64.2 |
| Glu/Gln | 40.7 | 43.6 | 40.8 | 41.6 | 93.0 |
| Ser | 11.2 | 11.3 | 11.3 | 12.7 | 33.3 |
| His | 5.3 | 5.3 | 5.4 | 5.0 | 14.6 |
| Gly | 9.1 | 9.3 | 9.2 | 9.4 | 37.8 |
| Thr | 9.0 | 9.3 | 9.2 | 9.5 | 29.8 |
| Arg | 14.1 | 14.1 | 14.2 | 13.6 | 44.8 |
| Ala | 10.5 | 9.9 | 10.4 | 9.9 | 33.7 |
| Tyr | 5.5 | 5.3 | 5.4 | 5.5 | 16.8 |
| Val | 12.3 | 10.3 | 10.1 | 10.0 | 28.0 |
| Met | 4.8 | 4.8 | 5.2 | 5.3 | 9.1 |
| Phe | 10.3 | 10.4 | 10.3 | 10.1 | 23.8 |
| Ile | 8.6 | 8.6 | 8.7 | 8.3 | 25.4 |
| Leu | 18.1 | 17.1 | 18.0 | 16.4 | 48.9 |
| Lys | 14.1 | 15.2 | 14.8 | 14.3 | 47.3 |
| Pro | 11.0 | 12.2 | 11.0 | 11.8 | 22.7 |
| Sum | 207.4 | 209.4 | 207.0 | 205.7 | 573.3 |

### Measurements

The birds were weighed on the arrival day altogether and average initial body weight was determined. Pen based feed intake and individual body weight development were measured at daily and weekly intervals, respectively. Pen based average body weight (BW), daily weight gain (ADG), feed intake (FI), DM intake (DMI), and feed conversion ratio (FCR, i.e., feed:gain ratio) were calculated in the end of each period. During the last 6 days of P2, all birds were evaluated daily for the presence of sticky faeces (SF) attached to cloaca, indicating the effect of dietary NSP on faeces consistency. A total of 10 additional birds in the first experimental run (n=240 +10) were also necropsied to sample on d 1 for basal measurements (i.e. sampled for digesta and plasma). Mortality was recorded daily.

On days 8 and 16 (i.e. end of P1 and P2, respectively), randomly selected animals (n=15 birds per group were sampled for tissue and digesta from ileum and caeca. A colon sample (in most cases >200 mg) was collected. The birds sampled for digesta were also measured for full weights of small intestine and caeca. For small intestine only jejunum and ileum weights were recorded together, but duodenum was excluded as a precise separation of pancreas was not possible because of time reasons during slaughter. The residual egg-yolk-sac attached to the small intestine at Meckel's diverticulum was not removed and weighed together with jejunum and ileum. Caeca were cut from the junction point with ileum and colon and the full weight of the caecum pairs was determined collectively. Tissue samples from ileum and caeca were also collected and stored in RNA or formalin. Slaughter blood was collected from all birds and plasma was separated, stored at −20° C. for later use.

Preparation of earthworms to be fed to Group 3. Based on the feed consumption on the previous day, the amount of worms to be given the next day were determined. For this purpose, daily DM intake of Group 3 (average of 6 pens) was calculated (I). Earthworms were given as 1% of DM intake of the previous day.

Worms were given on DM-basis as 1% of DMI of the previous day. DMI of the previous day=(Feed intake×0.9)+(fresh worm intake×0.15). The coefficients (0.9 and 0.15) are the approximate correction factors for DM of feed and worms, respectively.

The earthworms used in the experiment belong to the European nightcrawler, i.e. Dendrobaena veneta (sometimes referred to as Eisenia hortensis). The amount of worms (given as fresh substrate) provided to the birds thus corresponded to ca. 6.7% of total DMI of the previous day, respectively. All 6 pens of Group 3 received the same amount of earthworms. The living earthworms were separated from the rearing soil material (provided by the earthworm rearing company, i.e., “Martin Langhoff SUPERWURM e.K.”), weighed, cut in a few pieces by using a pair of scissors and offered to the birds on a feed plate (ca. 30 cm in diameter). Small size worms were used for the study. Overall average weight of the earthworms used in the study was 157 mg per worm (SD=47; n=36 measurements on randomly selected batches of 13-37 worms). Approximate time spent by the birds of Group 3 for consuming worms (n=6 pens) were assessed. For this purpose, time was recorded when the worms were offered. Thereafter, the pens in which worms were offered were frequently observed (ca. every 2 to 10 min, depending on the amount of worms left on the plates) and approximate time for consuming all the worms was then determined.

### Statistical Analyses

Data were analyzed with analysis of variance using the GLM procedure of SAS (V9.4). Data from each period were analyzed separately. The experimental unit for growth (e.g. average bird weight in a pen, average ADG of birds in a pen), feed intake and litter moisture parameters were the pen. Similarly, percentage of animals with SF and time spent consuming worms were calculated based on pen data. For intestinal size measurements, bird-individual measurements were used (i.e. a replicate was a bird). For all the pen-based data, total number of replicates throughout 2 runs was N=48. Except for CON+ diet in period 1 (n=12), numbers of pens per diet was n=6 in both periods 1 and 2, respectively, in each run.

Statistical model for pen based variables included fixed effects of diet, run, diet×run and blocking effects of room plus residual random error. For the bird-individual data, the statistical model additionally included blocking effects of pens. For the time spent consuming worms, the statistical model included only day and room effects. No run effect was included in this model, because full data were available only from the second run. Similarly, no day×room interaction was included in the model, because in two rooms there was only one pen receiving worms, as shown in table 2.

Least square means (LSMEANs) were separated using the Tukey test. Group differences were considered significant at P<0.05, and tend to differ at P<0.10. Data are presented as LSEMANS and their SE. For the sake of a succinct presentation, only the most conservative SE (i.e. the largest) of LSMEANS is presented. Since the n numbers of replicates for all groups were the same in the second period (n=6), SEs of LSMEANS were identical for this period.

### Mortality

Overall mortality across all group, periods and experimental runs was ca. 1.1%. Average overall mortality per diet and period across two runs is summarized in Table 5.

TABLE 5
Mortality (%) in birds receiving experimental
diets across two runs (N = 480).

| Diet | Period 1 | Period 2 |
|---|---|---|
| CON+ | 2.5 | 1.7 |
| CON− | n/a | 0.0 |
| CON + VC | 1.7 | n/a |
| CON − VC | n/a | 0.0 |
| CON + EW | 0.0 | n/a |
| CON − EW | n/a | 1.7 |
| Av. Mort. | 1.4 | 0.8 |

N/a = not applicable as the CON− diet was fed only in P2.

It is evident from table 5 that the morality rate was reduced with inventive feed composition or the vermicompost feed composition.

### Bird Growth, Feed Intake and Feed Conversion Efficiency

Treatment effects resulted in significant differences in ADG, WG and BW of the birds consuming different diets in P1 (P<0.05; Table 4). As compared to CON+ diet, CON+VC improved (P<0.05) ADG, WG and BW in P1, through an elevated feed intake (P<0.05; Table with no effect on FCR (P>0.05). CON+EW did not differ from the CON+ in terms of growth and feed intake (P>0.05) in P1, whereas CON+VC tended to (P<0.10) result in higher growth and feed intake than CON+EW in P1. Excluding contribution of worm intake by Group 3, DMI tended to be higher (P=0.099) in CON+VC than in CON+ or CON+EW. Overall DMI through feed and worm intake was however not significantly different among 3 diets in P1 (P=0.121, Table 5).

In P2, CON− did not affect growth or DMI relative to CON+ (P>0.05), although CON− birds consumed ca. 6% higher amount of feed than did those birds on CON+ diet. In the same period (P2), CON−VC fed birds consumed a numerically (P=0.106) higher amount of feed (ca. +7%), tended to have a higher ADG (P=0.084) and were still heavier (P<0.05) than those birds fed CON+ (Table 4 and 5). Birds on CON−EW did not differ (P>0.05) from those on CON− or CON−VC diets in terms of growth, feed conversion efficiency and feed intake parameters.

TABLE 4
Pen-based average body weight (BW), weight gain (WG) and daily weight gain (ADG) of
broiler birds receiving experimental diets in two different periods of 8 days each*.

| | Diets | | | | | P-values | | | |
|---|---|---|---|---|---|---|---|---|---|
| Period 1 | CON+ | CON− | CON + EW | CON + VC | SE | Diet | Run | Dxr | Room |
| BW, g/bird | 192^{a} | n/a | 193^{ab†} | 203b^{†} | 2.97 | 0.019 | 0.001 | 0.926 | 0.632 |
| WG, g/bird | 151^{a} | n/a | 153^{ab†} | 162b^{†} | 2.90 | 0.022 | 0.028 | 0.957 | 0.668 |
| ADG, g/bird | 18.9^{a} | n/a | 19.1^{ab†} | 20.2b^{†} | 0.36 | 0.022 | 0.028 | 0.957 | 0.668 |
| Period 2 | CON+ | CON− | CON − EW | CON − VC | | | | | |
| BW, g/bird | 549^{a} | 580^{ab} | 578^{ab} | 600^{b} | 11.63 | 0.036 | 0.001 | 0.856 | 0.047 |
| WG, g/bird | 360 | 385 | 385 | 397 | 10.02 | 0.084 | 0.004 | 0.829 | 0.035 |
| ADG, g/bird | 45.0 | 48.1 | 48.1 | 49.6 | 1.25 | 0.084 | 0.004 | 0.829 | 0.035 |

*Periods 1 and 2 include animals at the age of “day-0 to day-7” and “day-8 to day-15”, respectively.
^{ab}Groups denoted with different letters differ significantly (Tukey, P < 0.05). The sign (†) indicates tendency to differ (Tukey, P = 0.103).
Abbreviations: CON+: positive control diet; CON−: negative control diet; CON + EW: positive control diet plus 1% of earthworm; CON + VC: positive control diet supplemented with 1% vermicompost; CON − EW: negative control diet plus 1% earthworm; CON − VC: negative control diet supplemented with 1% vermicompost. BW: average body weight in a pen, WG: average weight gain within a period; ADG: average daily gain within a period; DxR: Diet and Run interaction.
N/a = not applicable as the CON− diet was fed only in P2.

TABLE 5
Feed and worm intakes and feed conversion efficiency estimates for broiler birds
receiving experimental diets in two different periods of 8 days each *.

| | Diets | | | | | P-values | | | |
|---|---|---|---|---|---|---|---|---|---|
| Period 1 | CON+ | CON− | CON + EW | CON + VC | SE | Diet | Run | Dxr | Room |
| Feed | 176^{a} | n/a | 176ab^{†} | 186b^{†} | 2.94 | 0.025 | 0.208 | 0.764 | 0.507 |
| intake, | | | | | | | | | |
| g/bird | | | | | | | | | |
| FCR_1 | 1.16 | n/a | 1.15 | 1.15 | 0.019 | 0.807 | 0.001 | 0.838 | 0.700 |
| DMI_feed, | 161 | n/a | 161 | 168 | 2.69 | 0.099 | 0.088 | 0.775 | 0.506 |
| g/bird | | | | | | | | | |
| DMI_worm, | 0 | n/a | 1.22 | 0 | — | — | — | — | — |
| g/bird | | | | | | | | | |
| DMI, g/bird | 161 | n/a | 162 | 168 | 2.69 | 0.121 | 0.086 | 0.770 | 0.505 |
| FCR_2 | 1.07 | n/a | 1.06 | 1.04 | 0.018 | 0.483 | 0.001 | 0.824 | 0.696 |
| Period 2 | CON+ | CON− | CON − EW | CON − VC | | | | | |
| Feed | 465 | 494 | 483 | 501 | 10.69 | 0.106 | 0.062 | 0.819 | 0.072 |
| intake, | | | | | | | | | |
| g/bird | | | | | | | | | |
| FCR_1 | 1.30 | 1.29 | 1.26 | 1.27 | 0.030 | 0.806 | 0.099 | 0.967 | 0.672 |
| DMI_feed, | 425 | 454 | 443 | 456 | 9.77 | 0.122 | 0.116 | 0.806 | 0.073 |
| g/bird | | | | | | | | | |
| DMI_worm, | 0 | 0 | 3.89 | 0 | — | — | — | — | — |
| g/bird | | | | | | | | | |
| DMI, g/bird | 425 | 454 | 447 | 456 | 9.76 | 0.122 | 0.112 | 0.789 | 0.073 |
| FCR_2 | 1.19 | 1.18 | 1.17 | 1.15 | 0.028 | 0.843 | 0.055 | 0.965 | 0.678 |

abGroups denoted with different letters differ significantly (Tukey, P < 0.05). The sign (†) indicates tendency of two diets to differ (Tukey, P = 0.063)
*Periods 1 and 2 include animals at the age of “day-0 to day-7” and “day-8 to day-15”, respectively.
Data are presented as LSMEANS and their SE. For the sake of a succinct presentation, only the most conservative SE (i.e. the largest) is presented. Since the n numbers for all groups were the same in the second period (n = 6), SEs of LSMEANS were identical for this period.
Abbreviations: CON+: positive control diet; CON−: negative control diet; CON + EW: positive control diet supplemented with 1% of earthworm; CON + VC: positive control diet supplemented with 1% vermicompost; CON − EW: negative control diet supplemented with 1% earthworm; CON − VC: negative control diet supplemented with 1% vermicompost; Feed intake: Total feed intake in a given period, g/ bird; FCR_1: Feed:weight gain ratio, g/g; DMI_feed; dry matter intake of birds in a given period, g/bird. DMI_feed: dry matter intake through feed, g/bird; DMI_worm: DM intake through worm consumption; DMI: total dry matter intake, g/bird; FCR_2: DMI: weight gain ratio, g/g.
N/a = not applicable as the CON− diet was fed only in P2.

### Small Intestine (SI) and Caeca Weights

Consuming earthworms tended to (P=0.081) increase proportion of jejunum+ileum+residual egg yolk sac (i.e. JI-ResEYS) to BW ratio without any significant effect on the weight of JI-ResEYS (P=0.491) in P1 (Table 6). As compared to CON+, caeca weight tended (P=0.054) to be increased by CON+VC in P1, which then disappeared in P2 (P>0.05). In contrast, caeca weight of earthworm consuming birds in P1 (i.e. CON+EW) did not differ from CON+ fed birds, whereas EW increased caeca weight in combination with CON− diet in P2 (P=0.031). CON− did not induce any significant effect on JI-ResEYS or caeca weights when fed alone (i.e. CON−) or supplemented with vermicompost (i.e. CON−VC) in P2 (P<0.05).

TABLE 6
Absolute weights of small intestine (jejunum + ileum + residual
egg yolk sac; JI-ResEYS) and caeca and their proportion to body weights
(BW) in broiler birds fed different diets in the end of two periods of 8 days each.

| | Diets | | | | | P-values | | | |
|---|---|---|---|---|---|---|---|---|---|
| Period 1 | CON+ | CON− | CON + EW | CON + VC | SE | Diet | Run | DxR | Room |
| JI-ResEYS, g | 15.57 | n/a | 16.08 | 16.10 | 0.441 | 0.491 | 0.031 | 0.358 | 0.582 |
| JI-ResEYS, | 7.75 | n/a | 8.12 | 747 | 0.143 | 0.081 | 0.127 | 0.244 | 0.543 |
| % of BW | | | | | | | | | |
| Caeca, g | 2.57 | n/a | 2.69 | 2.91 | 0.118 | 0.054 | 0.809 | 0.816 | 0.258 |
| Caeca % of | 1.28 | n/a | 1.37 | 1.42 | 0.055 | 0.114 | 0.37 | 0.671 | 0.116 |
| BW | | | | | | | | | |
| Period 2 | CON+ | CON− | CON − EW | CON − VC | | | | | |
| JI-ResEYS, g | 36.59 | 39.16 | 37.73 | 39.9 | 1.274 | 0.249 | 0.137 | 0.357 | 0.314 |
| JI-ResEYS, | 6.56 | 6.63 | 6.54 | 6.24 | 0.252 | 0.69 | 0.099 | 0.763 | 0.415 |
| % of BW | | | | | | | | | |
| Caeca, g | 6.57 | 6.90 | 7.73 | 7.36 | 0.288 | 0.031 | 0.001 | 0.377 | 0.293 |
| Caeca % of | 1.18 | 1.18 | 1.33 | 1.2 | 0.061 | 0.256 | 0.071 | 0.693 | 0.478 |
| BW | | | | | | | | | |

Periods 1 and 2 include animals at the age of “day-0 to day-7” and “day-8 to day-15”, respectively.
ab: Groups denoted with different letters differ significantly (Tukey, P < 0.05). The sign (†) indicates tendency to differ (Tukey, P = 0.103).
Data are presented as LSEMANS and their SE. For the sake of a succinct presentation, only the most conservative SE (i.e. the largest) is presented. Since the n numbers replicates for all groups were the same in the second period (n = 6), SEs of LSMEANS were identical for this period.
Abbreviations: CON+: positive control diet; CON−: negative control diet; CON + EW: positive control diet supplemented with 1% of earthworm; CON + VC: positive control diet supplemented with 1% vermicompost; CON − EW: negative control diet supplemented with 1% earthworm; CON − VC: negative control diet supplemented with 1% vermicompost.
n/a = not applicable as the CON− diet was fed only in P2.

Replicates: A bird was considered as replicate (N=273 birds, i.e., n=15-35 birds/group in each period and run). Litter moisture and sticky feces

Parameters assessing moisture content and moisture accumulation in the litter did not differ among the experimental diets in either period (P>0.05; Table 7). Although proportion of moisture accumulated in litter was numerically the lowest in CON+ (6.2%-) and highest in CON−VC (8.0%), differences were not significant until end of P2 (P>0.05).

The overall average frequency of SF across all diets was negligible (2.6%) in P1 (notice that SF was evaluated in all days of R2, whereas it was assessed only during the last 6 days of R1, thus results presented from this point onwards refer to the last 6 days of both runs, i.e. see FIG. 2). In the end of P2, 10% of CON+ birds had SF (FIG. 2). As compared to CON+, CON− diet (P<0.05) increased prevalence of SF (40.5%) and VC aggravated this effect (57.9%), whereas CON−EW (18.9%) did not differ from CON+ (P>0.05). As compared with CON− feeding, CON−EW tended to decrease (P=0.072) prevalence of SF. No significant run effect was observed on the frequency of SF (P=0.071) and there was no significant interaction of treatment and run effects on SF prevalence (P=0.730; FIG. 2).

TABLE 7

| | Diets | | | | | P-values | | | |
|---|---|---|---|---|---|---|---|---|---|
| Period 1 | CON+ | CON− | CON + EW | CON + VC | SE | Diet | Run | DxR | Room |
| Dry | 90.2 | n/a | 90.2 | 88.7 | 1.15 | 0.516 | 0.559 | 0.502 | 0.403 |
| matter % | | | | | | | | | |
| Moisture % | 9.8 | n/a | 9.8 | 11.3 | 1.15 | 0.516 | 0.559 | 0.502 | 0.403 |
| Water | 7.2 | n/a | 7.3 | 8.8 | 1.15 | 0.516 | 0.559 | 0.502 | 0.403 |
| accumulated % | | | | | | | | | |
| Period 2 | CON+ | CON− | CON − EW | CON − VC | | | | | |
| Dry | 91.3 | 90.5 | 90.1 | 89.5 | 1.01 | 0.628 | 0.308 | 0.566 | 0.816 |
| matter % | | | | | | | | | |
| Moisture % | 8.7 | 9.5 | 9.9 | 10.5 | 1.01 | 0.628 | 0.308 | 0.566 | 0.816 |
| Water | 6.2 | 6.9 | 7.4 | 8.0 | 1.01 | 0.628 | 0.308 | 0.566 | 0.816 |
| accumulated % | | | | | | | | | |

*: Periods 1 and 2 include animals at the age of “day-0 to day-7” and “day-8 to day-15”, respectively. Litter samples were taken in the end of each period and were air dried at 70° C.

Basal dry matter of clean, unused litter (i.e. wood shaving) was 97.4%. Moisture accumulated in the litter until end of a period was calculated as the difference of dry matter in basal sample and sampled litter.

Abbreviations: CON+: positive control diet; CON−: negative control diet; CON+EW: positive control diet supplemented with 1% of earthworm; CON+VC: positive control diet supplemented with 1% vermicompost; CON−EW: negative control diet supplemented with 1% earthworm; CON−VC: negative control diet supplemented with 1% vermicompost. DxR: Diet and Run interaction, n/a=not applicable as the CON− diet was fed only in P2.

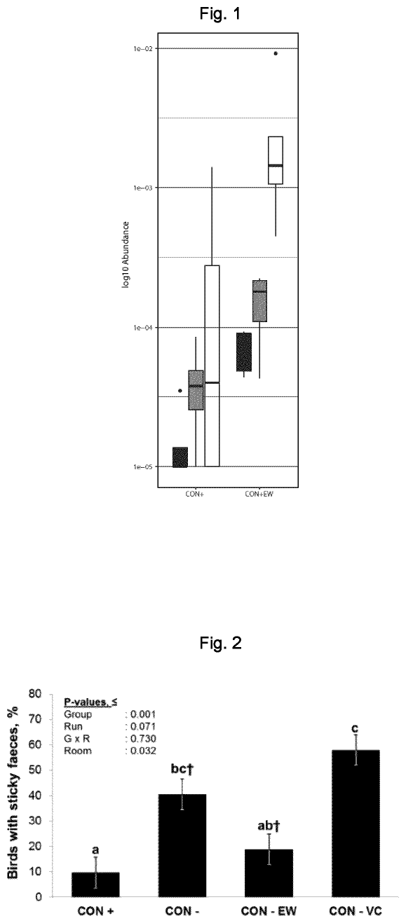

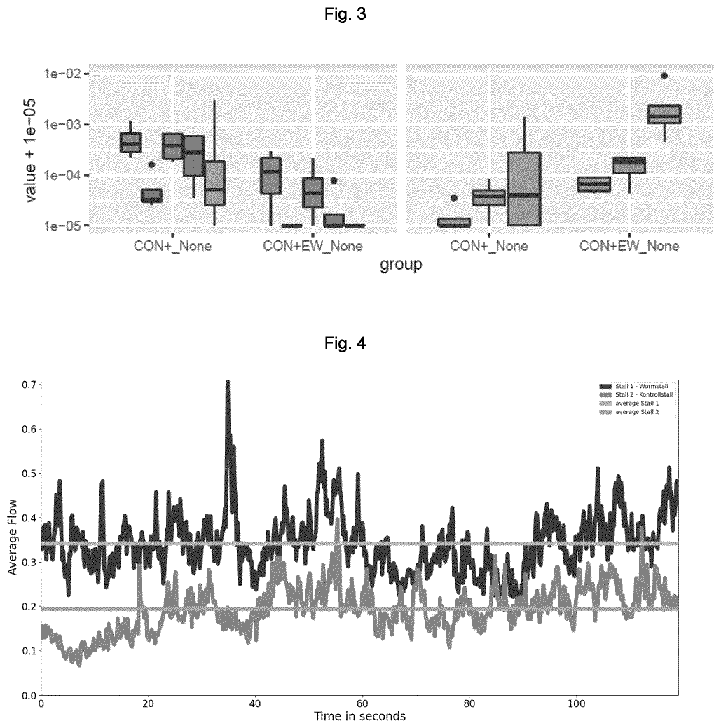

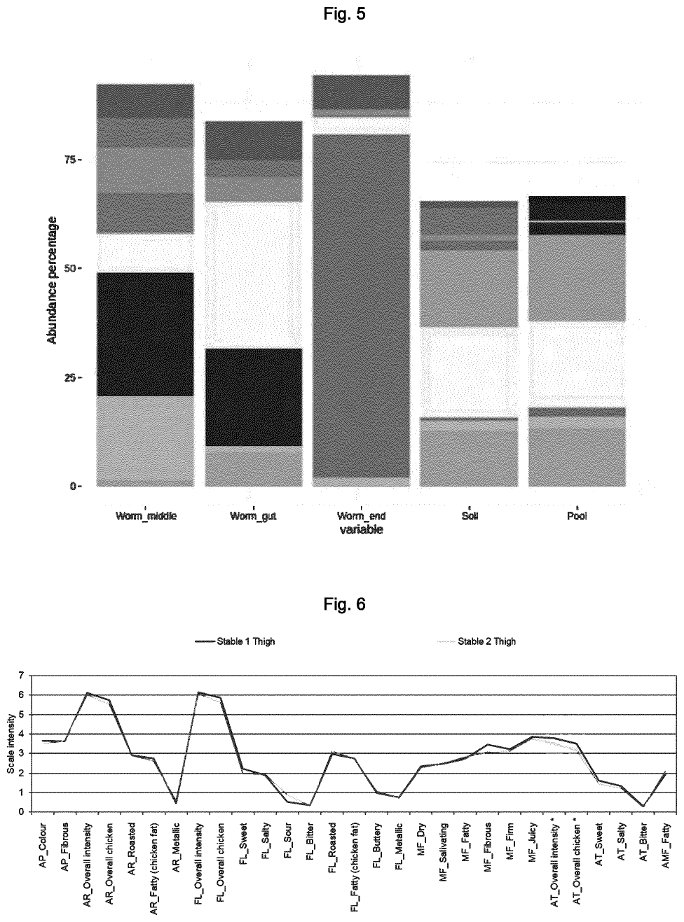

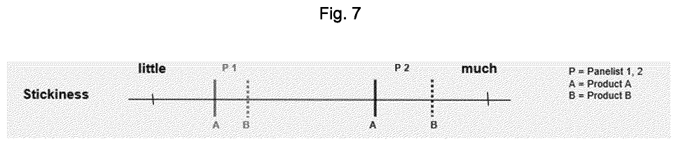

Transferring complex microbiota to young chicken in early life is a means to make them more resilient towards pathogens. The use of earth worms (Dendrobena veneta) provide a) such complex microbiota and b) an attractive feed for the young chicken. In order to confirm a change in microbiota, feces samples were collected and characterized via standard PCR testing. The complex microbiota is characterized via 16S bacterial community analysis. The method includes isolation of DNA from chicken digesta or feces followed by 16S rRNA gene amplification and NGS sequencing. FIG. 1 shows that some of the important (Akkermansia muciniphilia, Lactobacillus coleohominis and Bifidobacterium pseudolongum) gut inhabitants increased in the feces of stable 1 due to feeding of worms-vermicompost-mix over the days 2 to 6 of the fattening period.

Transferring complex microbiota to young chicken is a means to make them more resilient towards pathogens in early life. The specific pathogen free worms are able to provide a) such complex microbiota and b) an attractive feed for the young chicken.

In this trial the effect of SPF worm and vermicompost mixture was evaluated. The parameter is regularly screened for in German slaughterhouses in order to evaluate the animal welfare status of the chicken. This is performed by cameras which give an objective result even at the high speed of the processing line. The method is approved by the State Veterinary Service. Two commercial broiler houses were started off with 16000 birds of the ROSS 308 line each. Stables were fully comparable: same structure, same feeding and drinking systems, same climate, same bedding, same feed, same bird origin (hatchery).

Stable 2 received a mix of Earth Worms (Dendrobena Veneta) and Vermicompost (1/1 weight) in the following amounts (amounts in g):
Day 1: 2 g mix per bird
Day 2-3: 0 g mix per bird
Day 4 to day 18: 0.4 g mix per bird

After 32 days 7000 birds were randomly selected and slaughtered from both stable and classified in the slaughterhouse according to their level of pododermatits (PD) by camera. All 4 classes of pododermatites (PD) were evaluated and is shown in table 6.

Table 6 shows the distribution in % of PD between the two stables

| Pododermatitis in % | Stable 1 | Stable 2 |
|---|---|---|
| (PD classes 0/1/2a/2b) | (control) % | % |
| PD 0 | 25 | 49 |
| PD 1 | 5 | 42 |
| PD 2a | 53 | 7 |
| PD 2b | 17 | 2 |

PD 0 class means no pododermatitis and PD 2b means sever pododermatitis.
It is evident from above table that the chicken fed with SPF earthworm and vermicompost mixture reduced the incidence of pododermatitis in chicken remarkably.

In this experiment two commercial broiler houses were started off with 16,000 birds of the ROSS 308 line each. Stables were fully comparable: same structure, same feeding and drinking systems, same climate, same bedding, same feed, same bird origin (hatchery). Stable 1 received a mix of Earth Worms (Dendrobena Veneta) and Vermicompost (1/1 weight) in the following amounts and days:

TABLE 7

| Day of | Worms, |
|---|---|
| fattening | kg/d |
| 2 | 4 |
| 3 | 4.5 |
| 4 | 5 |
| 5 | 6.5 |
| 6 | 8 |

On day 40^{th} the birds were slaughtered, and 10 tibias were prepared from randomly selected birds from each stable. The tibiae were tested for various properties as shown in table 8.

TABLE 8

| | Stable 1 | | Stable 2 | | P- |
|---|---|---|---|---|---|
| Measurement | Mean | SD | Mean | SD | value |
| Peak load (N) | 290 | 65.7 | 280 | 70.0 | 0.746 |
| Displacement (mm) | 3.97 | 0.575 | 4.13 | 0.596 | 0.549 |
| Modulus (Mpa) | 134 | 24.3 | 118 | 29.5 | 0.196 |
| Work to failure (J) | 0.631 | 0.207 | 0.633 | 0.210 | 0.981 |
| Fresh tibia weight (g) | 11.13 | 1.686 | 11.85 | 1.904 | 0.384 |
| Dry tibia weight (g) | 5.64 | 0.914 | 5.88 | 0.881 | 0.550 |
| Dry matter (%) | 50.6 | 1.86 | 49.7 | 1.86 | 0.305 |
| Ash percentage (dry tibia) | 44.7 | 1.71 | 43.4 | 2.01 | 0.149 |
| Ash weight (g/tibia) | 2.51 | 0.389 | 2.56 | 0.418 | 0.813 |

Table 8 shows various measures, of which Modulus and ash percentage direct to the trend of improvement in tibiae of worm-fed birds, indicating better bone strength due to more active behaviour compared to the other.

In another experiment complex microbiota to young chicken was transferred to the young chicken. This means to make them more resilient towards pathogens in early life. Stable 2 was fed with earth worms (Dendrobena veneta) to provide a) such complex microbiota and b) an attractive feed for the young chicken. the two commercial broiler houses were started off with 16,000 birds of the ROSS 308 line each. Stables were fully comparable: same structure, same feeding and drinking systems, same climate, same bedding, same feed, same bird origin (hatchery). Stable 2(table 9) received a mix of Earth Worms (Dendrobena Veneta) and Vermicompost (1/1 weight) in the following amounts and days:

TABLE 9

| Day | Worms, kg |
|---|---|
| 14 | 4.0 |
| 15 | 4.5 |
| 16 | 5.0 |
| 17 | 6.5 |
| 18 | 8.0 |

The chicken in the control stable suffered from an unspecified infection and were treated with antibiotic as of day 19 of fattening while the worm-fed stable (stable 2) did not show symptoms of infection and was not treated.

TABLE 10
Losses during the fattening period and at the slaughterhouse

| | Stable 1 | Stable 2 |
|---|---|---|
| | control | worms |
| Losses, stable % | 6.2 | 2.84 |
| Condemnation | 5.68 | 1.51 |

It is evident from table 10 that the chickens which received worms in week 3 of the fattening period were not suffering from the infection. This is expressed by reduced losses in the stable and at the slaughterhouse.

Started off with 10.000 in stable 1 and with 8.000 birds in the control stable 2. The chicken line was Hobbard slow growing breed. Stables were fully comparable: same structure, same feeding and drinking systems, same climate, same bedding, same feed, same bird origin (hatchery). Stable 1 received a mix of Earth Worms (Dendrobena Veneta) and Vermicompost (1/1 weight) in the following amounts (amounts of worms in g):

TABLE 11

| Day | Worms Kg |
|---|---|
| 2 | 2.2 |
| 3 | 2.2 |
| 4 | 2.5 |
| 5 | 3.3 |
| 6 | 4.1 |

After 20 days the samples were taken by walking the identical ways through the stables with boot-socks. The socks were sent for llumina sequencing of the pen-floor metagenomics. FIG. 2 shows that some of the important (Akkermansia muciniphilia, Lactobacillus coleohominis and Bifidobacterium pseudolongum) gut inhabitants increased in the feces of stable 1 due to feeding of worms-vermicompost-mix over the days 2 to 6 of the fattening period. At the same time, unwanted bacteria like Blautia. Clostridium piliformis and Citrobacter are significantly reduced by worm-vermicompost-mix feeding.

### Preparation of Cold Water Vermicompost Extract

20 g of the vermicompost was weighed and added to 80 mL of distilled water and stirred. The vermicompost was filtrated off using a clean cheesecloth and the solution was collected. A total of 2 g Hatch gel was added to the filtrate. Thus obtained solution was sprayed a volume of 0.25 mL per chicken. After spraying, the crate of chickens was placed in a well-lit environment for 15 minutes to facilitate preening.

The body weight (BW) is measured and recorded individually on D0, D14, D28, D35 and D42. Body weight is calculated using following formula:

Average Body Weight (BW) per group is calculated for D0, D14, D28, D35 and D42. The DWG at bird level is calculated using following formula for the birds completing the study:

$$\frac{( Weight D 14 - Weight D 0 )}{Total number of days study period}$$

The DWG at pen level is calculated using following formula:

$$\frac{( Pen weight D 14 + weight dead birds D 0 D 14 - Pen weight D 0 )}{total number of bird days}$$

The total number of bird days is the sum of the period that each individual bird participated in the study. For example, if 4 birds complete a study period of 8 days and 1 bird dies 1 day after allocation, the total number of bird days equals 4*8+1*1=33.

The total feed weight is measured per pen. On D0, D14, D28, and D35, the feed provided to the birds is measured (FEED IN) and recorded on FOR-TEST-012 (Feed weight). When on any other day a pen would be running out of feed and more feed would need to be provided, the added feed is also be weighed and recorded as “FEED IN”. On D14, D28, D35 and D42, the weight of the remaining feed is measured (FEED OUT) and noted on FOR-TEST-012 (Feed weight). The daily feed intake is calculated per pen using following formula:

$$Average daily feed intake ( g ) = \frac{( FEED IN - FEED OUT )}{Total number of bird days}$$

The feed conversion ratio (FCR) is calculated at pen level for each of the above mentioned study periods, using following formula:

$$Feed conversion ratio = \frac{Average daily feed intake}{Average daily weight gain}$$

TABLE 12

| | | Mean | Mean | Mean DFI |
|---|---|---|---|---|
| Treatment | Group name | DWGD0D42 | BWD42 | D0D42 |
| T01 | control | 75.7 | 3226 | 102.3 |
| T11 | Spray-on VC - 1:5 | 77.1 | 3282 | 106.2 |

The effects of feeding earthworm on the chicken meat quality in the end of the process was evaluated. The approach for the evaluation included 10 panelists and 2 measurements over a period of 6 days. The method included descriptive analysis (QDA type), the intensities assessed with a 10-point unstructured line scale, assessment accuracy 0.1. There was 2 training session and 1 measurement session and was blinded for product order per session. The statistical analysis of the result was based on Univariate Analysis of Variance and Post Hoc LSD; significance level p≤0.05. The Descriptive Analysis (QDA type) delivers a complete profile of each product covering all sensory dimensions by using a specifically discussed and commonly understood list of attributes developed by the panel. A 10 cm line scale is used to rate the intensity of each attribute. For training suitable references are used, here samples of the product set. Panel is not calibrated to specific intensities per attribute, they use rankings (B higher than A) as shown in FIG. 7. Two assessments per product were conducted. The chicken line was Ross 308 and the stable 1 was fed with earth worms and stable 2 was without feeding with earth worms. Thighs were used for the evaluation. Overall, 28 attributes are applicable, and 28 attributes discriminate the products as shown in table 13.

TABLE 13

| | | | | After- | After |
|---|---|---|---|---|---|
| Appearance | Odour | Flavour | Mouthfeel | taste | Mouthfeel |
| Colour | Overall | Overall | Dry | Overall | Fatty |
| | intensity | intensity | | intensity | |
| Fibrous | Overall | Overall | Salivating | Overall | |
| | Chicken | Chicken | | Chicken | |
| | Roasted | Sweet | Fatty | Sweet | |
| | Fatty | Salty | Fibrous | Salty | |
| | (Chicken | | | | |
| | fat) | | | | |
| | Metallic | Sour | Firm | | |
| | | Bitter | Juicy | | |
| | | Roasted | | | |
| | | Fatty | | | |
| | | (Chicken | | | |
| | | fat) | | | |
| | | Buttery | | | |
| | | Metallic | | | |

Upon comparison it is evident form FIG. 6 that that the meat quality of the chicken fed with earthworm had a higher overall intensity and overall aftertaste.

1. A feed composition comprising at least one living worm,
wherein the worm is free of pathogens selected from the group consisting of species of Salmonella, species of Campylobacter, Histomonas meleagridis and any combination of two or more of the aforementioned, and/or SPF free according to Ph. Eur. 9, Ausgabe, Grundwerk 2017, 9.0/5.02.02.00.

2. (canceled)

3. The feed composition according to claim 1, wherein the at least one living worm in the composition is present in an amount in the range of 40 wt. % to 60 wt. % based on the total wet weight of the composition.

4. The feed composition according to claim 1, wherein the composition further comprises vermicompost.

5. The feed composition according to claim 4, wherein the vermicompost is present in an amount in the range of 40 wt. % to 60 wt. % based on the total wet weight of the composition.

6. The feed composition according to claim 1, further comprising:
vermicompost, wherein the vermicompost is free of pathogens selected from the group consisting of species of Salmonella, species of Campylobacter, Histomonas meleagridis and any combination of two or more of the aforementioned.

7. A process for feeding animals for non-therapeutic use comprising feeding the animal with the composition according to claim 1 in an amount in the range of 2 mg to 50 mg living worm/g body weight of the animal per day.

8. The process according to claim 7, wherein the animal is selected from the group consisting of chicklet, piglet, lambs, calves, duckling, and gosling.

9.-14. (canceled)

15. A method of producing vermicompost comprising the steps of:
a. raising worms from pathogen free breeding stock;
b. feeding the worms with pathogen free feed;
c. keeping/raising the worms in pathogen free surroundings; and
d. collecting the vermicompost,
wherein “pathogen free” is free of pathogens selected from the group consisting of species of Salmonella, species of Campylobacter, Histomonas meleagridis and any combination of two or more of the aforementioned.

16. A pathogen free vermicompost obtained according to the method of claim 15.

17. A process for preparing a liquid vermicompost extract comprising the steps of:
a. adding water to the vermicompost obtained according to the method of claim 15 to obtain a mixture,
b. agitating the mixture obtained in step a. for a period of 1 minute to 1 hour, and
c. filtering to collect a filtrate.

18. The process according to claim 17, wherein the water is free of chlorine and has a temperature in the range of 5-20° C.

19. A liquid vermicompost extract obtained according to the process of claim 17.

20. A process of transferring complex microbiota to an animal comprising
spraying the liquid vermicompost extract according to claim 19 onto the animals,
or delivering to the animals via drinking water.

21. (canceled)

22. (canceled)

23. The feed composition according to claim 1, wherein the feed composition is used to obtain a young animal having higher counts of gut bacteria in their feces via standard PCR testing.

24. The feed composition according to claim 1, wherein the feed composition is used to obtain an animal with increased weight.

25. The pathogen free vermicompost according to claim 16, wherein the pathogen free vermicompost is used for raising an animal having higher counts of gut bacteria in their feces characterized via standard PCR testing.

26. The liquid vermicompost extract according to claim 19, wherein the vermicompost extract is used to obtain an animal having higher counts of gut bacteria in their feces characterized via standard PCR testing.

27. The liquid vermicompost extract according to claim 19, wherein the vermicompost extract is used to obtain an animal free of pathogens,
wherein the pathogens are selected from the group consisting of species of Salmonella, species of Campylobacter, Histomonas meleagridis and any combination of two or more of the aforementioned.

28. The feed composition according to claim 1 or the specific pathogen free vermicompost according to claim 16 or the liquid vermicompost extract according to claim 19 used to reduce the number or the intensity of feet (pododermatitis) lesions in an animal.

29. The pathogen free vermicompost according to claim 16 to reduce the number or the intensity of feet lesions in an animal.
